(19) United States
(12) Patent Application Publication
(10) Pub. No.: US 2026/0028594 A1
(43) Pub. Date: Jan. 29, 2026

(54) CELL PATCH CRYOPRESERVATION SOLUTION AND METHOD OF CRYOPRESERVING A CELL PATCH

(71) Applicants: BOE REGENERATIVE MEDICINE TECHNOLOGY CO., LTD., Chaoyang District, Beijing (CN); BOE TECHNOLOGY GROUP CO., LTD., Beijing (CN); BEIJING BOE TECHNOLOGY DEVELOPMENT CO., LTD., Beijing (CN)

(72) Inventors: Juan WANG, Beijing (CN); Shuang GAO, Beijing (CN); Lu SHI, Beijing (CN); Zhixin ZHANG, Beijing (CN); Donghua LIU, Beijing (CN); Shuai LIU, Beijing (CN)

(21) Appl. No.: 18/994,327

(22) PCT Filed: Apr. 20, 2023
(86) PCT No.: PCT/CN2023/089575

§ 371 Date: Jan. 14, 2025

(51) Int. Cl. C12N 5/00

(52) U.S. Cl. CPC C12N 5/525; C12N 5/526

(57) ABSTRACT

The present disclosure provides a cell patch cryopreservation solution, which includes: a cryoprotectant, a buffer, a reducing agent, a calcium channel inhibitor and an apoptosis inhibitor. The present disclosure also provides a method of cryopreserving a cell patch by using the aforementioned cell patch cryopreservation solution. The cell patch cryopreservation solution and the method of cryopreserving a cell patch provided by the present disclosure can maintain the structural integrity and the cell activity of the cell patch.

### TECHNICAL FIELD

The present invention is in a field of cell biology. In particular, the present invention relates to a cell patch cryopreservation solution and a method of cryopreserving a cell patch.

### BACKGROUND

A cell patch is a layered structure composed of multiple layers of cells with extracellular matrix secreted by the cells. The cell patch is a more efficient way of transplanting stem cells, and the cell patch can effectively prevent the loss of stem cells during the transplantation process and improve the efficiency of transplanting the stem cells compared to the conventional method of transplanting the stem cells by injecting stem cells in suspension. In addition, the cell patch is prepared without using enzymes and the like for cell digestion, which effectively avoids the destruction of the extracellular matrix and the reduction of the cell function caused by the enzyme digestion, and the cell patch transplantation enables the stem cells to perform their functions better in vivo.

Currently, the cell patches are typically applied in a ready-to-use manner, which requires that the patient needs to make an appointment at least one week in advance before using it. Also, the cell patches have a limited storage time in a storage solution, typically not exceeding 24 hours, and after preparation, it needs to be used for the patient as soon as possible. In addition, some classical safety-related tests, such as sterility tests and mycoplasma tests, cannot obtain results before using it due to the short window period of use, so that rapid detection methods or risk release are required, which increases the risk of use the cell patches and the cost of detection.

Therefore, the development of cell patch cryopreservation technology is of great significance in transforming cell patches from ready to use products to shelf products. Different from the traditional single cell suspension cryopreservation, the cell patch cryopreservation requires overcoming two major challenges: 1) ensuring cell viability at a high cell density per unit volume, and 2) ensuring that cell-to-cell connections are not disrupted. Traditional and commercially available cell cryopreservation solutions are not able to successfully cryopreserve cell suspensions, which are characterized by the disadvantage that the cell patch structure is disrupted and the low cell survival rate after cryopreservation and resuscitation.

Therefore, there is a need to develop a cell patch cryopreservation solution and a method of cryopreserving a cell patch that guarantees the structural integrity and cellular activity of the cell patches.

### SUMMARY

The present invention aims to solve one of the technical problems in the related art at least to some extent. An aspect of the present disclosure provides a cell patch cryopreservation solution, which comprises: a cryoprotectant, a buffer, a reducing agent, a calcium channel inhibitor, and an apoptosis inhibitor.

For example, the apoptosis inhibitor is at least one selected from a group consisting of a P53 inhibitor and a Caspase inhibitor. Optionally, a molar concentration of the apoptosis inhibitor is 1 to 50 M based on the whole cell patch cryopreservation solution. In one embodiment, the apoptosis inhibitor is the P53 inhibitor with a molar concentration of 1 to 50 μM or the Caspase inhibitor with a molar concentration of 1 to 50 μM, based on the whole cell patch cryopreservation solution.

For example, the calcium channel inhibitor is at least one selected from a group consisting of anipamil, falipamil, devapamil, gallopamil, tiapamil and verapamil. Optionally, a molar concentration of the calcium channel inhibitor is 5 to 500 M based on the whole cell patch cryopreservation solution. In one embodiment, the calcium channel inhibitor is verapamil with a molar concentration of 5 to 500 M based on the whole cell patch cryopreservation solution.

For example, the reducing agent is at least one selected from a group consisting of VC with a mass concentration of 1 to 100 μg/mL, reduced glutathione with a molar concentration of 0.3 to 30 mM, allopurinol with a molar concentration of 0.1 to 10 mM, adenylate with a molar concentration of 0.5 to 50 mM, based on the whole cell patch cryopreservation solution.

For example, the cryoprotectant is at least one selected from a group consisting of DMSO with a volume concentration of 2% to 10%, sucrose with a mass concentration of 2 g/100 mL to 10 g/100 mL, ethylene glycol with a volume concentration of 1% to 5%, cross-linked polyvinylpyrrolidone with a mass concentration of 2 g/100 mL to 10 g/100 mL, propylene glycol with a volume concentration of 2% to 10%, trehalose with a mass concentration of 2 g/100 mL to 10 g/100 mL, methylcellulose with a mass concentration of 0.05 g/100 mL to 0.5 g/100 mL, glycerol with a volume concentration of 5% to 15% based on the whole cell patch cryopreservation solution.

For example, the buffer comprises one of a HEPES buffer system, a phosphate buffer system, a MOPS buffer system, a PIPES buffer system.

Optionally, the cell patch cryopreservation solution further comprises a nutritional additive, and the nutritional additive comprises transferrin, amino acids and glucose. For example, the amino acid is at least one selected from a group consisting of glycine with a molar concentration of 0.4 to 1.2 mM, alanine with a molar concentration of 0.2 to 0.6 mM, arginine with a molar concentration of 0.3 to 2.4 mM, aspartate with a molar concentration of 0.1 to 0.7 mM, aspartic acid with a molar concentration of 0.05 to 0.5 mM, cysteine with a molar concentration of 0.3 to 0.9 mM, cystine with a molar concentration of 0.05 to 0.3 mM, glutamic acid with a molar concentration of 0.3 to 0.9 mM, glutamine with a molar concentration of 1 to 3 mM, histidine with a molar concentration of 0.1 to 0.8 mM, isoleucine with a molar concentration of 0.2 to 1.0 mM, leucine with a molar concentration of 0.2 to 1.0 mM, lysine with a molar concentration of 0.2 to 1.0 mM, methionine with a molar concentration of 0.05 to 0.5 mM, phenylalanine with a molar concentration of 1.1 to 0.6 mM, proline with a molar concentration of 0.1 to 0.6 mM, serine with a molar concentration of 0.1 to 0.6 mM, threonine with a molar concentration of 0.2 to 1.0 mM, tryptophan with a molar concentration of 0.01 to 0.15 mM, tyrosine with a molar concentration of 0.1 to 0.6 mM, valine with a molar concentration of 0.2 to 1.0 mM, based on the whole cell patch cryopreservation solution. Optionally, the nutritional additive further comprises at least one selected from a group consisting of fetal bovine serum, bovine serum albumin, human serum albumin and platelet lysate.

In one implementation scheme of the cell patch cryopreservation solution, the cryoprotectant is DMSO with a volume concentration of 5% and methyl cellulose with a mass concentration of 0.1 g/100 mL; the buffer system is a HEPES buffer system; the nutritional additive comprises human serum albumin with a mass concentration of 1 g/100 mL, glucose with a mass concentration of 1000 g/mL, transferrin with a mass concentration of 1 g/mL, glycine with a molar concentration of 0.7 mM, alanine with a molar concentration of 0.3 mM, arginine with a molar concentration of 0.6 mM, aspartate with a molar concentration of 0.3 mM, aspartic acid with a molar concentration of 0.2 mM, cysteine with a molar concentration of 0.6 mM, cystine with a molar concentration of 0.1 mM, glutamic acid with a molar concentration of 0.5 mM, glutamine with a molar concentration of 2 mM, histidine with a molar concentration of 0.2 mM, isoleucine with a molar concentration of 0.4 mM, leucine with a molar concentration of 0.4 mM, lysine with a molar concentration of 0.4 mM, methionine with a molar concentration of 0.1 mM, phenylalanine with a molar concentration of 0.2 mM, proline with a molar concentration of 0.3 mM, serine with a molar concentration of 0.2 mM, threonine with a molar concentration of 0.4 mM, tryptophan with a molar concentration of 0.05 mM, tyrosine with a molar concentration of 0.2 mM, and valine with a molar concentration of 0.4 mM; the reducing agent is VC with a mass concentration of 10 g/mL; the calcium channel inhibitor is verapamil with a molar concentration of 30 M; the apoptosis inhibitor is the Caspase inhibitor Q-VD-OPh with a molar concentration of 10 M, based on the whole cell patch cryopreservation solution.

In another implementation scheme of the cell patch cryopreservation solution, the cryoprotectant is DMSO with a volume concentration of 10%; the buffer system is a phosphate buffer system; the nutritional additive comprises bovine serum albumin with a mass concentration of 1 g/100 mL, glucose with a mass concentration of 1000 g/mL, transferrin with a mass concentration of 1 g/mL, glycine with a molar concentration of 0.7 mM, alanine with a molar concentration of 0.3 mM, arginine with a molar concentration of 0.6 mM, aspartic acid with a molar concentration of 0.3 mM, aspartic acid with a molar concentration of 0.2 mM, cysteine with a molar concentration of 0.6 mM, cystine with a molar concentration of 0.1 mM, glutamic acid with a molar concentration of 0.5 mM, glutamine with a molar concentration of 2 mM, histidine with a molar concentration of 0.2 mM, isoleucine with a molar concentration of 0.4 mM, leucine with a molar concentration of 0.4 mM, lysine with a molar concentration of 0.4 mM, methionine with a molar concentration of 0.1 mM, phenylalanine with a molar concentration of 0.2 mM, proline with a molar concentration of 0.3 mM, serine with a molar concentration of 0.2 mM, threonine with a molar concentration of 0.4 mM, tryptophan with a molar concentration of 0.05 mM, tyrosine with a molar concentration of 0.2 mM, and valine with a molar concentration of 0.4 mM; the reducing agent is VC with a mass concentration of 5 μg/mL and reduced glutathione with a molar concentration of 3 mM; the calcium channel inhibitor is verapamil with a molar concentration of 20 μM; the apoptosis inhibitor is the Caspase inhibitor Q-VD-OPh with a molar concentration of 10 μM, based on the whole cell patch cryopreservation solution.

In still another implementation scheme of the cell patch cryopreservation solution, the cryoprotectant is DMSO with a volume concentration of 10%; the buffer system is a MOPS buffer system; the nutritional additive comprises platelet lysate with a volume concentration of 1%, glucose with a mass concentration of 1200 μg/mL, transferrin with a mass concentration of 5 μg/mL, glycine with a molar concentration of 0.7 mM, alanine with a molar concentration of 0.3 mM, arginine with a molar concentration of 0.6 mM, aspartic acid with a molar concentration of 0.3 mM, aspartic acid with a molar concentration of 0.2 mM, cysteine with a molar concentration of 0.6 mM, cystine with a molar concentration of 0.1 mM, glutamic acid with a molar concentration of 0.5 mM, glutamine with a molar concentration of 2 mM, histidine with a molar concentration of 0.2 mM, isoleucine with a molar concentration of 0.4 mM, leucine with a molar concentration of 0.4 mM, lysine with a molar concentration of 0.4 mM, methionine with a molar concentration of 0.1 mM, phenylalanine with a molar concentration of 0.2 mM, proline with a molar concentration of 0.3 mM, serine with a molar concentration of 0.2 mM, threonine with a molar concentration of 0.4 mM, tryptophan with a molar concentration of 0.05 mM, tyrosine with a molar concentration of 0.2 mM, and valine with a molar concentration of 0.4 mM; the reducing agent comprises VC with a mass concentration of g/mL, reduced glutathione with a molar concentration of 3 mM, and allopurinol with a molar concentration of 1 mM; the calcium channel inhibitor is verapamil with a molar concentration of 25 μM; the apoptosis inhibitor is the P53 inhibitor Pifithrin-α with a molar concentration of 10 μM, based on the whole cell patch cryopreservation solution.

Another aspect of the present disclosure provides a method of cryopreserving a cell patch, which comprises: contacting a cell patch with any one of the cell patch cryopreservation solutions mentioned above; and cryopreserving the cell patch with the cell patch cryopreservation solution. Optionally, the cell patch cryopreservation solution is pre-cooled at 2 to 8° C. before contacting with the cell patch.

Another aspect of the present disclosure provides a composition, which comprises any one of the cell patch cryopreservation solutions mentioned above and a cell patch.

The cell patch cryopreservation solution and the method of cryopreserving the cell patch in the present disclosure are able to better maintain the structural integrity and cellular viability of the cell patch.

### BRIEF DESCRIPTION OF DRAWINGS

In order to more clearly explain the technical solution of the embodiments of the present disclosure, the accompanying drawings of the embodiments will be briefly introduced below. Obviously, the accompanying drawings in the following description only relate to some embodiments of the present disclosure, and are not limited to the present disclosure.

FIG. 1 is images of appearances of a cell patch before cryopreservation by using the cell patch cryopreservation solution and after resuscitation of Example 1, the left image shows before cryopreservation, and the right image shows after resuscitation.

FIG. 2 is images of the appearances of the cell patches after resuscitation by using the cell patch cryopreservation solutions of Examples 2, 3, and 4, respectively.

FIG. 3 is images of the appearances of the cell patches after resuscitation by using the cell patch cryopreservation solutions of Comparative Example 1 and Comparative Example 2, the left image shows the Comparative Example 1 and the right image shows the Comparative Example 2.

FIG. 4 shows cell survival rates of the cell patches after cryopreservation and resuscitation by using the cell patch cryopreservation solutions of Examples 1, 2, 3 and Comparative Examples 1, 2 respectively.

FIG. 5 shows cell survival rates of the cell patches after cryopreservation and resuscitation by using the cell patch cryopreservation solutions of Examples 1, 4 and Comparative Examples 5, 6, 7.

### DETAILED DESCRIPTION

In order to make the purpose, technical solution and advantages of the embodiment of the present disclosure clearer, the technical solution of the embodiment of the present disclosure will be described clearly and completely with the accompanying drawings. Obviously, the described embodiment is a part of the embodiment of the present disclosure, not the whole embodiment. Based on the described embodiments of the present disclosure, all other embodiments obtained by ordinary skilled in the art without creative labor belong to the scope of protection of the present disclosure.

The present disclosure may be embodied in other specific forms without departing from the essential attributes thereof. It is to be understood that, any and all embodiments of the present invention may be combined with technical features of any other embodiment or embodiments to obtain additional embodiments without conflict. The present invention includes additional embodiments resulting from such combinations.

All publications and patents mentioned in the present disclosure are hereby incorporated by reference into the present disclosure in their entirety. If the usage or terminology used in any one of the publications and patents incorporated by reference conflicts with the usage or terminology used in the present disclosure, the usage and terminology of the present disclosure shall prevail.

The chapter titles used in the present disclosure are only for the purpose of organizing the present disclosure and should not be interpreted as a limitation on the subject matter.

Unless otherwise specified, all technical and scientific terms used herein have the usual meaning of the present disclosure to which the claimed subject matter belongs. If there are a plurality of definitions for a term, the definitions in the present disclosure shall prevail.

Except as indicated in the work examples or otherwise, all numbers expressing quantitative properties such as dosages set forth in the specification and claims are to be understood as being modified in all instances by the term “about.” It should also be understood that, any numerical range recited in the present application is intended to include all sub-ranges within such range and any combination of the individual endpoints of such range or sub-range.

The use of words such as “including”, “containing” or “comprising” and the like in the present disclosure means that the appearance of elements preceding the word encompasses the elements listed after the word and their equivalents, but does not exclude unrecited elements. The term “containing” or “comprising” as used herein may be open-ended, semi-closed and closed-ended. In other words, the term also includes “essentially consisting of” or “consisting of”.

A cell patch is a layered structure composed of multiple layers of cells, cells are connected to each other through extracellular matrix secreted by the cells. The cell patch is opalescent and semi-transparent in appearance with smooth surface and clean edges. Cell patches can be obtained by a variety of methods, for example, the cell patches are formed by stimulating extracellular matrix (ECM) secretion by Vitamin C, the cell patches are formed by coating culture dishes with temperature-sensitive materials, light-sensitive materials, electrically-sensitive materials, or polymeric human fibrin and so on, as described in the review of “Application of Cell Patch Technology in Tissue Engineering” in the Chinese Journal of Cells and Stem Cells, Volume 7, Issue 6, December 2017.

The cell patch of the present application is an animal cell patch, preferably a mammalian cell patch, and still more preferably a human derived cell patch. Human derived cell patches include, for example, the human umbilical cord mesenchymal stem cell patch, but may be other cell patch types. The cell patch may be of various sizes, for example, the cell patch may have a thickness of 20 μm to 300 μm.

The cell patch cryopreservation solution of the present disclosure comprises: a cryoprotectant, a buffer, a reducing agent, a calcium channel inhibitor, and an apoptosis inhibitor. The cell patch cryopreservation solution of the present disclosure may be obtained by mixing the above components.

Cryoprotectants are divided into non-permeating cryoprotectants and permeating cryoprotectants according to whether they penetrate the cell patch. The non-permeating cryoprotectants, for example, include sucrose, cross-linked polyvinylpyrrolidone, trehalose, methylcellulose, polyethylene glycol, dextran, hydroxyethyl starch, and the like, are impermeable to the cell patches but can increase the osmotic pressure of the extracellular fluid, thereby allowing intracellular water to enter the extracellular fluid and reducing the formation of intracellular ice crystals. The permeating cryoprotectants can penetrate the cell patches and enter the cells to exert their effects, which has a stronger direct effect on the cells than the non-permeating cryoprotectants. For example, dimethyl sulfoxide (DMSO), ethylene glycol, propylene glycol, glycerol, and the like are small molecule substances that bind to water molecules through hydrogen bonding in the cells to inhibit the formation of the ice crystal, which reduces the crystallization temperature of the cryoprotectants, and forms a glass transition state from a semi-solid state to a solid state at the low temperature.

In one embodiment, the cryoprotectant comprises at least one selected from a group consisting of DMSO with a volume concentration of 2% to 10%, sucrose with a mass concentration of 2 g/100 mL to 10 g/100 mL, ethylene glycol with a volume concentration of 1% to 5%, cross-linked polyvinylpyrrolidone with a mass concentration of 2 g/100 mL to 10 g/100 mL, propylene glycol with a volume concentration of 2% to 10%, trehalose with a mass concentration of 2 g/100 mL to 10 g/100 mL, methylcellulose with a mass concentration of 0.05 g/100 mL to 0.5 g/100 mL, glycerol with a volume concentration of 5% to 15% based on the whole cell patch cryopreservation solution. Calculated based on the whole cell patch cryopreservation solution, the cryoprotectant is DMSO with a volume concentration of 10% and methylcellulose with a mass concentration of 0.1 g/100 mL. For example, calculated based on the whole cell patch cryopreservation solution, the cryoprotectant is DMSO with a volume concentration of 10%.

The buffer may be selected from biological buffers conventionally used in cell culture, for example, a HEPES buffer system, a phosphate buffer system, a MOPS buffer system, and a PIPES buffer system.

Reducing agents are used to protect cells from damaging caused by reactive oxygen species, such as reduced glutathione, VC, allopurinol, adenylate.

In one embodiment, the reducing agent is at least one selected from a group consisting of VC with a mass concentration of 1 to 100 μg/mL, reduced glutathione with a molar concentration of 0.3 to 30 mM, allopurinol with a molar concentration of 0.1 to 10 mM, adenylate with a molar concentration of 0.5 to 50 mM, based on the whole cell patch cryopreservation solution. For example, the reducing agent is VC with a mass concentration of 10 μg/mL based on the whole cell patch cryopreservation solution. As another example, the reducing agents are VC with a mass concentration of 5 μg/mL and reduced glutathione with a molar concentration of 3 mM based on the whole cell patch cryopreservation solution. As another example, the reducing agents are VC with a mass concentration of 5 μg/mL, reduced glutathione with a molar concentration of 3 mM, and allopurinol with a molar concentration of 1 mM based on the whole cell patch cryopreservation solution.

Calcium channel inhibitors are agents that block the trans-membrane transport of calcium ions across calcium channels on the cell patch. In one embodiment, the calcium channel inhibitor is at least one selected from a group consisting of anipamil, falipamil, devapamil, gallopamil, tiapamil and verapamil. The concentration of the calcium channel inhibitor is typically with a molar concentration of 5-500 M based on the whole cell patch cryopreservation solution. For example, the calcium channel inhibitor is verapamil with a molar concentration of 30 μM, 20 μM or 25 μM based on the whole cell patch cryopreservation solution. It is believed that the calcium channel inhibitors in the cell patch cryopreservation solution may prevent cell apoptosis caused by changes in calcium ion concentration by hindering changes in calcium ion concentration in the cell.

An apoptosis inhibitor is an agent capable of inhibiting apoptosis of a cell. The inventors have found that the addition of specific apoptosis inhibitors (for example P53 inhibitors, caspase inhibitors) to the cell patch cryopreservation solution can improve the survival rate of the cryopreserved cells after resuscitation. In one embodiment, the apoptosis inhibitor in the cell patch cryopreservation solution comprises at least one of P53 inhibitor and the Caspase inhibitor. The Caspase inhibitor is for example Q-VD-Oph (CAS number: 1135695-98-5, available from Abeam). An example of the P53 inhibitor is Pifithrin-alpha (commercially available from stemgent, Inc., and the molecular formula is Ci₆HisN₂OS-HBr).

Preferably, the apoptosis inhibitor has a molar concentration of 1-50 M based on the whole cell patch cryopreservation solution. For example, the apoptosis inhibitor is the P53 inhibitor with a molar concentration of 1-50 M or the Caspase inhibitor with a molar concentration of 1-50 M based on the whole cell patch cryopreservation solution.

In one embodiment, the apoptosis inhibitor in the cell patch cryopreservation solution is the P53 inhibitor with a molar concentration of 10 M based on the whole cell patch cryopreservation solution. In one embodiment, the apoptosis inhibitor in the cell patch cryopreservation solution is the Caspase inhibitor with a molar concentration of 10 M based on the whole cell patch cryopreservation solution.

The cell patch cryopreservation solution in the present disclosure also optionally comprises a nutritional additive including transferrin, amino acids, and glucose. In one embodiment, the amino acid is at least one selected from a group consisting of glycine with a molar concentration of 0.4 to 1.2 mM, alanine with a molar concentration of 0.2 to 0.6 mM, arginine with a molar concentration of 0.3 to 2.4 mM, aspartic acid with a molar concentration of 0.1 to 0.7 mM, aspartic acid with a molar concentration of 0.05 to 0.5 mM, cysteine with a molar concentration of 0.3 to 0.9 mM, cystine with a molar concentration of 0.05 to 0.3 mM, glutamic acid with a molar concentration of 0.3 to 0.9 mM, glutamine with a molar concentration of 1 to 3 mM, histidine with a molar concentration of 0.1 to 0.8 mM, isoleucine with a molar concentration of 0.2 to 1.0 mM, leucine with a molar concentration of 0.2 to 1.0 mM, lysine with a molar concentration of 0.2 to 1.0 mM, methionine with a molar concentration of 0.05 to 0.5 mM, phenylalanine with a molar concentration of 1.1 to 0.6 mM, proline with a molar concentration of 0.1 to 0.6 mM, serine with a molar concentration of 0.1 to 0.6 mM, threonine with a molar concentration of 0.2 to 1.0 mM, tryptophan with a molar concentration of 0.01 to 0.15 mM, tyrosine with a molar concentration of 0.1 to 0.6 mM, valine with a molar concentration of 0.2 to 1.0 mM, based on the whole cell patch cryopreservation solution.

Optionally, the nutritional additive further comprises at least one of fetal bovine serum, bovine blood albumin, human blood albumin and platelet lysate. In one embodiment, the cell patch cryopreservation solution does not contain the bovine serum, the bovine blood albumin, the human blood albumin, or the platelet lysate.

The present disclosure also provides a method of cryopreserving a cell patch, and the method comprises: contacting a cell patch with any one of the above-mentioned cell patch cryopreservation solutions, and cryopreserving the cell patch with the cell patch cryopreservation solution.

Contacting the cell patch with any one of the above-mentioned cell patch cryopreservation solutions, for example, may be by submerging the cell patch in the cryopreservation solution.

Preferably, the cell patch cryopreservation solution is pre-cooled at 2-8° C. before contacting with the cell patch.

The present disclosure further provides a method of resuscitating a cell patch, and the method comprises:

The cryopreserved cell patch comprising the cell patch and the above-mentioned cell patch cryopreservation solution was resuscitated in a constant temperature apparatus at 35-39° C., and then the resuscitated cell patch was transferred to a culture dish to which PBS or physiological saline was added in advance, the liquid is removed from the culture dish and PBS or physiological saline is added into the culture dish.

The cell patch cryopreservation solution and the method of cryopreserving the cell patch in the present disclosure are able to better maintain the structural integrity and cellular viability of the cell patch, and the cell survival rate of the cell patch after resuscitation is above 80%.

The present invention will now be further described with reference to the following examples, but the present invention is not limited to the following examples. Where no specific conditions are noted in the examples, they were carried out according to conventional conditions or those suggested by the manufacturer. Any reagents or instruments used that do not indicate the manufacturer are commercially available conventional products.

### (1) Formulation of the Cell Patch Cryopreservation Solution

The cell patch cryopreservation solution was obtained by mixing the components of the following formulation. As an exception, the commercial cryopreservation solution in the Comparative Example 2 was outsourced.

### Example 1 (Formulation 1)

Cryoprotectant: DMSO with a volume concentration of 5%, methylcellulose with a mass concentration of 0.1 g/100 mL.

### Buffer System: HEPES Buffer System.

Nutritional additives: human serum albumin with a mass concentration of 1 g/100 mL (by adding 5% of the total formulation volume of human serum albumin with a mass concentration of 20 g/100 ml), glucose with a mass concentration of 1000 μg/mL, transferrin with a mass concentration of 1 μg/mL and amino acid. The amino acid was glycine with a molar concentration of 0.7 mM, alanine with a molar concentration of 0.3 mM, arginine with a molar concentration of 0.6 mM, aspartate with a molar concentration of 0.3 mM, aspartic acid with a molar concentration of 0.2 mM, cysteine with a molar concentration of 0.6 mM, cystine with a molar concentration of 0.1 mM, glutamic acid with a molar concentration of 0.5 mM, glutamine with a molar concentration of 2 mM, histidine with a molar concentration of 0.2 mM, isoleucine with a molar concentration of 0.4 mM, leucine with a molar concentration of 0.4 mM, lysine with a molar concentration of 0.4 mM, methionine with a molar concentration of 0.1 mM, phenylalanine with a molar concentration of 0.2 mM, proline with a molar concentration of 0.3 mM, serine with a molar concentration of 0.2 mM, threonine with a molar concentration of 0.4 mM, tryptophan with a molar concentration of 0.05 mM, tyrosine with a molar concentration of 0.2 mM, and valine with a molar concentration of 0.4 mM. The amino acids were prepared as a 100-fold concentrated storage solution of amino acid combinations by using injection water. The volume of the amino acids added during preparation was 1% of the total formulation.

Reducing agent: VC with a mass concentration of 10 μg/mL. VC was prepared into a 1000-fold concentrated storage solution with injection water, which had a mass concentration of 10 mg/mL. The volume of the VC added during preparation was 0.1% of the total formulation.

Calcium channel inhibitor: Verapamil with a molar concentration of 30 μM. Verapamil was prepared into a 1000-fold concentrated storage solution with injection water, which had a molar concentration of 30 mM. The volume of the verapamil added during preparation was 0.1% of the total formulation.

Apoptosis inhibitor: Caspase inhibitor Q-VD-Oph (CAS number: 1135695-98-5, purchased from Abeam), with a molar concentration of 10 μM. The Caspase inhibitor Q-VD-Oph was formulated as a 1000-fold concentrated storage solution, which had a molar concentration of 10 μM. The caspase inhibitor Q-VD-Oph was prepared in DMSO as a 1000-fold concentrated storage solution, which had a molar concentration of 10 mM. The volume of the caspase inhibitor added during preparation was 0.1% of the total formulation.

### Example 2 (Formulation 2)

Cryoprotectant DMSO accounts for 10% of the total formulation volume.

### Buffer System: Phosphate Buffer System.

Nutritional additives: bovine serum albumin with a mass concentration of 1 g/100 mL (by adding 5% of the total formulation volume of bovine serum albumin with a mass concentration of 20 g/100 ml), glucose with a mass concentration of 1000 μg/mL (prepared into a 100-fold concentrated storage solution with injection water, the volume of the glucose added during preparation was 1% of the total formulation), transferrin with a mass concentration of 1 μg/mL (prepared into a 1000-fold concentrated storage solution with injection water, the volume of the transferrin added during preparation was 0.1% of the total formulation) and amino acid. The amino acid was glycine with a molar concentration of 0.7 mM, alanine with a molar concentration of 0.3 mM, arginine with a molar concentration of 0.6 mM, aspartate with a molar concentration of 0.3 mM, aspartic acid with a molar concentration of 0.2 mM, cysteine with a molar concentration of 0.6 mM, cystine with a molar concentration of 0.1 mM, glutamic acid with a molar concentration of 0.5 mM, glutamine with a molar concentration of 2 mM, histidine with a molar concentration of 0.2 mM, isoleucine with a molar concentration of 0.4 mM, leucine with a molar concentration of 0.4 mM, lysine with a molar concentration of 0.4 mM, methionine with a molar concentration of 0.1 mM, phenylalanine with a molar concentration of 0.2 mM, proline with a molar concentration of 0.3 mM, serine with a molar concentration of 0.2 mM, threonine with a molar concentration of 0.4 mM, tryptophan with a molar concentration of 0.05 mM, tyrosine with a molar concentration of 0.2 mM, and valine with a molar concentration of 0.4 mM (the amino acids were prepared as a 100-fold concentrated storage solution of amino acids combinations by using injection water, and the volume of the amino acids added during preparation was 1% of the total formulation).

Reducing agent: VC with a mass concentration of 5 μg/mL (VC was prepared into a 1000-fold concentrated storage solution with injection water, the volume of the VC added during preparation was 0.1% of the total formulation), reduced glutathione with a molar concentration of 3 mM (the reduced glutathione was prepared as a 1000-fold concentrated storage solution by using injection water, and the volume of the reduced glutathione added during preparation was 0.1% of the total formulation).

Calcium channel inhibitor: verapamil with a molar concentration of 20 M (verapamil was prepared into a 1000-fold concentrated storage solution with injection water, the volume of the Verapamil added during preparation was 0.1% of the total formulation).

Apoptosis inhibitor: Caspase inhibitor Q-VD-Oph (CAS number: 1135695-98-5, purchased from Abeam), with a molar concentration of 10 M. The Caspase inhibitor Q-VD-Oph was formulated as a 1000-fold concentrated storage solution, which had a molar concentration of 10 μM. The caspase inhibitor Q-VD-Oph was prepared in DMSO as a 1000-fold concentrated storage solution, which had a molar concentration of 10 mM. The volume of the caspase inhibitor added during preparation was 0.1% of the total formulation.

### Example 3 (Formulation 3)

Cryoprotectant DMSO accounts for 10% of the total formulation volume.

Buffer system: MOPS buffer system.

Nutritional additives: platelet lysate (accounts for 1% of the total formulation volume), glucose with a mass concentration of 1200 μg/mL, transferrin with a mass concentration of 5 μg/mL and amino acid. The amino acid was glycine with a molar concentration of 0.7 mM, alanine with a molar concentration of 0.3 mM, arginine with a molar concentration of 0.6 mM, aspartate with a molar concentration of 0.3 mM, aspartic acid with a molar concentration of 0.2 mM, cysteine with a molar concentration of 0.6 mM, cystine with a molar concentration of 0.1 mM, glutamic acid with a molar concentration of 0.5 mM, glutamine with a molar concentration of 2 mM, histidine with a molar concentration of 0.2 mM, isoleucine with a molar concentration of 0.4 mM, leucine with a molar concentration of 0.4 mM, lysine with a molar concentration of 0.4 mM, methionine with a molar concentration of 0.1 mM, phenylalanine with a molar concentration of 0.2 mM, proline with a molar concentration of 0.3 mM, serine with a molar concentration of 0.2 mM, threonine with a molar concentration of 0.4 mM, tryptophan with a molar concentration of 0.05 mM, tyrosine with a molar concentration of 0.2 mM, and valine with a molar concentration of 0.4 mM.

Reducing agent: VC with a mass concentration of 5 μg/mL, reduced glutathione with a molar concentration of 3 mM, and allopurinol with a mass concentration of 1 mM.

Calcium channel inhibitor: Verapamil with a molar concentration of 25 μM.

Apoptosis inhibitor: P53 inhibitor Pifithrin-α (Purchased from Stemdent Company, molecular formula was C₁₆H₁₈N₂OS·HBr) with a molar concentration of 10 M.

### Example 4 (Formulation 1—Human Serum Albumin)

The formulation of Example 4 was essentially the same as that of Example 1, except that human serum albumin was missing.

### Comparative Example 1 (FBS+DMSO

Formulation: FBS with a volume concentration of 90% (purchased from Gibco) and DMSO with a volume concentration of 10% (purchased from sigma).

### Comparative Example 2 (Commercial Cryopreservation Solution)

Name: MesenCult™-ACF Freezing Medium

Brand: STEMCELL technologies

Article number: 05490

### Comparative Example 3 (Formulation 1—Reducing Agent)

The formulation of Comparative Example 3 was essentially the same as that of Example 1, except that the reducing agent was missing.

### Comparative Example 4 (Formulation 1—Calcium Channel Inhibitor)

The formulation of Comparative Example 4 was essentially the same as that of Example 1, except that the calcium channel inhibitor was missing.

### Comparative Example 5 (Formulation 1—Apoptosis Inhibitor)

The formulation of Comparative Example 5 was essentially the same as that of Example 1, except that the apoptosis inhibitor was missing.

### (2) Cryopreservation of the Cell Patches

After obtaining the cell patches, the cell patches were washed once with PBS, pre-chilled cryopreservation solution at 2-8° C. was added, and the cell patches and the cryopreservation solution were transferred together to a cryopreservation tube. The cryopreservation tube was transferred to a gradient cooling box, then placed the cryopreservation tube in a −80° C. freezer overnight, and transferred to liquid nitrogen.

### (3) Resuscitation of the Cell Patches

The cryopreservation tube was rapidly melt in a 37° C. water bath until the ice crystals in the cryopreservation tube disappeared. The cell patches along with the cryopreservation solution were transferred to a culture dish previously added with PBS or physiological saline. The liquid was removed from the culture dish, and then PBS or physiological saline was added into the culture dish.

### (4) Study on the Morphology of the Cell Patches Before and After Cryopreservation.

Before the cryopreservation of the cell patches, the cell patches were placed and flattened in a 100 mm sterile culture dish, and morphological photos of the cell patches before cryopreservation were obtained by using a mobile phone to take photos.

After the cell patches recovery, the cryopreservation tube was rapidly melt in a 37° C. water bath until the ice crystals in the cryopreservation tube disappeared. The cell patches along with the cryopreservation solution were transferred to a culture dish with a volume of 100 mm previously added with PBS or physiological saline. The liquid was removed from the culture dish and the cell patches were flattened, and morphological photos of the cell patches after cryopreservation were obtained by using a mobile phone to take photos.

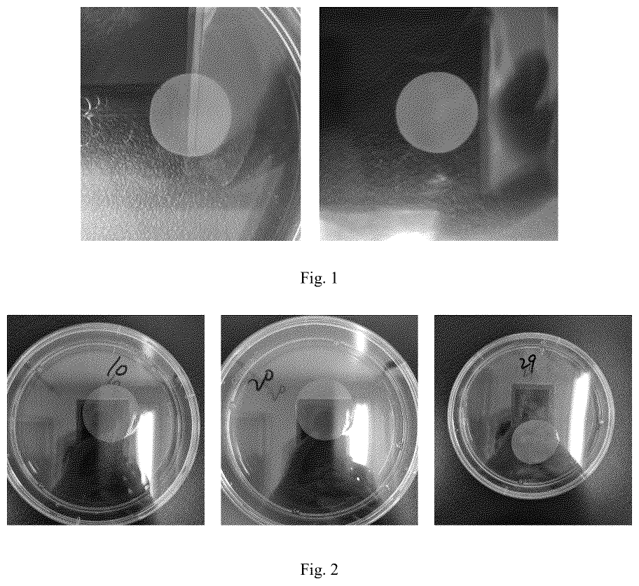

The left image in FIG. 1 is images of appearances of a cell patch before cryopreservation by using the cell patch cryopreservation solution of Example 1, the right image in FIG. 1 is a view of an appearance of a cell patch after cryopreservation and resuscitation by using the cell patch cryopreservation solution of Example 1. It can be seen from FIG. 1, the morphology of the cell patch was complete before and after cryopreservation, which indicates that compared to before cryopreservation, the survival rate of the cell patch after resuscitation does not decrease significantly. The experimental results for the cell patch cryopreservation solutions of Examples 2, 3 and 4 are similar to those of the cell patch cryopreservation solution of Example 1, as shown in FIG. 2.

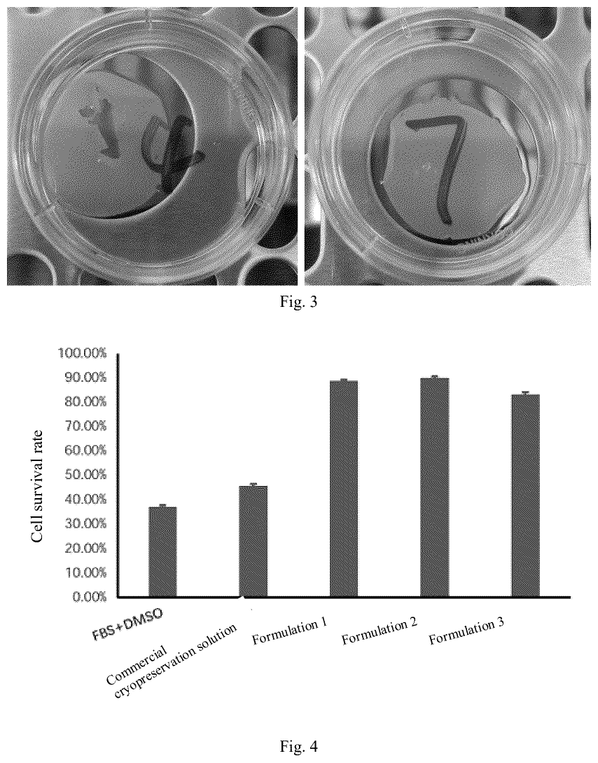

FIG. 3 is images of the appearances of the cell patches after resuscitation by using the cell patch cryopreservation solutions of the comparative example 1 (FBS and DMSO) and comparative example 2 (commercial cryopreservation solution). It can be seen from FIG. 3, the edges of the cell patch are not irregular and the morphology was incomplete.

### (5) Cell Counting and Survival Rate Detection

After the cell patches recovery, the liquid in the culture dish containing the cell patch was removed, the digestive enzyme TrypLE was added, the TrypLE and the cell patch were transferred to a 37° C. incubator, digested until the cell patch was loose, the digestion was stopped by adding the PBS, centrifuged, the supernatant was discarded, and the cell was counted and the survival rate of the cell was detected after the resuspension of PBS for cell precipitation.

The test method of the survival rate of the cell was as follows:
1) adding 3 mL TrypLE (purchased from Life technologies) to the human umbilical cord mesenchymal stem cell patch, and transferring them together to a sterile 50 mL centrifuge tube (purchased from Corning) and digesting for 3 minutes at 37° C.-5% carbon dioxide-saturated humidity incubator (purchased from ThermoFisher, model 371) until the cell patch of the human umbilical cord mesenchymal stem cell patch loosens.
2) adding 7 mL PBS to the centrifuge tube to dilute TrypLE, and using a 10 mL centrifuge tube or a 1 mL pipette to prepare single cells.
3) centrifuging the cell suspension at room temperature with a centrifugation speed of 300× g for 5 minutes (the centrifuge was purchased from ThermoFisher, and the model was ST16).
4) resuspending 10 mL of PBS for cell precipitation, and obtaining 100 μL of the PBS for cell counting and survival rate detection.
5) taking 3 sterile 1.5 mL EP tubes and adding 20 μL of AOPI (available from Countstar) to each of the sterile EP tubes.
6) adding 20 μL of the cell suspension to each of the 1.5 mL sterile EP tubes, mixing well, and taking 20 μL, then adding it to a cell counting plate.
7) inserting the cell counting plate into a fully automatic cell counter (purchased from Countstar, Model S2), and reading the cell survival rate, total cell density, and live cell density.

FIG. 4 compares the cell survival rates of the cell patches after resuscitation of the examples 1, 2, 3 and the comparative example 1 (FBS and DMSO) and the comparative example 2 (commercial cryopreservation solution). The cell survival rates of Examples 1, 2, and 3 were all above 80%, significantly higher than those of the comparative example 1 and the comparative example 2.

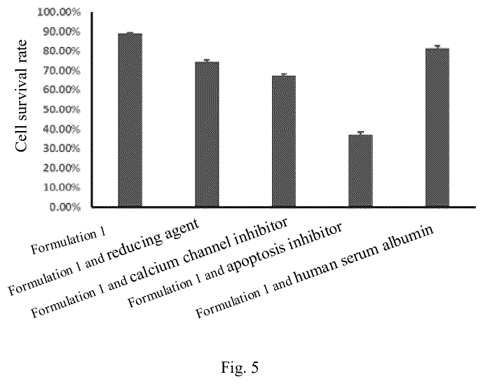

FIG. 5 compares the cell survival rates of the cell patches after resuscitation of the examples 1 and 4 and the comparative examples 3, 4, 5. It can be seen from FIG. 5, after removing the reducing agent, the calcium channel inhibitor, and the human serum albumin from the formulation of Example 1, the cell survival rates are affected, but the cell survival rate can still be maintained above 70%. Among them, example 4 (the formulation of the Example 1 removes the human serum albumin) has the smallest impact on the cell survival rate. However, the removal of the apoptosis inhibitor can significantly reduce the cell survival rate.

The above detailed description of the present invention is intended to enable those skilled in the art to understand the content of the present invention and implement it, and should not limit the protection scope of the present invention. Any equivalent changes or modifications made according to the spirit and essence of the present invention should be covered within the protection scope of the present invention.

1. A cell patch cryopreservation solution, comprising: a cryoprotectant, a buffer, a reducing agent, a calcium channel inhibitor and an apoptosis inhibitor, wherein the apoptosis inhibitor is at least one selected from a group consisting of a P53 inhibitor and a Caspase inhibitor.

2. The cell patch cryopreservation solution according to claim 1, wherein a molar concentration of the apoptosis inhibitor is 1 to 50 μM based on the whole cell patch cryopreservation solution.

3. The cell patch cryopreservation solution according to claim 1, wherein the apoptosis inhibitor is the P53 inhibitor with a molar concentration of 1 to 50 μM or the Caspase inhibitor with a molar concentration of 1 to 50 μM, based on the whole cell patch cryopreservation solution.

4. The cell patch cryopreservation solution according to claim 1, wherein the calcium channel inhibitor is at least one selected from a group consisting of anipamil, falipamil, devapamil, gallopamil, tiapamil and verapamil.

5. The cell patch cryopreservation solution according to claim 4, wherein a molar concentration of the calcium channel inhibitor is 5 to 500 μM based on the whole cell patch cryopreservation solution.

6. The cell patch cryopreservation solution according to claim 4, wherein the calcium channel inhibitor is verapamil with a molar concentration of 5 to 500 μM based on the whole cell patch cryopreservation solution.

7. The cell patch cryopreservation solution according to claim 1, wherein the reducing agent is at least one selected from a group consisting of VC with a mass concentration of 1 to 100 μg/mL, reduced glutathione with a molar concentration of 0.3 to 30 mM, allopurinol with a molar concentration of 0.1 to 10 mM, adenylate with a molar concentration of 0.5 to 50 mM, based on the whole cell patch cryopreservation solution.

8. The cell patch cryopreservation solution according to claim 1, wherein the cryoprotectant is at least one selected from a group consisting of DMSO with a volume concentration of 2% to 10%, sucrose with a mass concentration of 2 g/100 mL to 10 g/100 mL, ethylene glycol with a volume concentration of 1% to 5%, cross-linked polyvinylpyrrolidone with a mass concentration of 2 g/100 mL to 10 g/100 mL, propylene glycol with a volume concentration of 2% to 10%, trehalose with a mass concentration of 2 g/100 mL to 10 g/100 mL, methylcellulose with a mass concentration of 0.05 g/100 mL to 0.5 g/100 mL, glycerol with a volume concentration of 5% to 15% based on the whole cell patch cryopreservation solution.

9. The cell patch cryopreservation solution according to claim 1, wherein the buffer comprises one of a HEPES buffer system, a phosphate buffer system, a MOPS buffer system, a PIPES buffer system.

10. The cell patch cryopreservation solution according to claim 1, wherein the cell patch cryopreservation solution further comprises a nutritional additive, and the nutritional additive comprises transferrin, amino acids and glucose.

11. The cell patch cryopreservation solution according to claim 10, wherein the amino acid is at least one selected from a group consisting of glycine with a molar concentration of 0.4 to 1.2 mM, alanine with a molar concentration of 0.2 to 0.6 mM, arginine with a molar concentration of 0.3 to 2.4 mM, aspartate with a molar concentration of 0.1 to 0.7 mM, aspartic acid with a molar concentration of 0.05 to 0.5 mM, cysteine with a molar concentration of 0.3 to 0.9 mM, cystine with a molar concentration of 0.05 to 0.3 mM, glutamic acid with a molar concentration of 0.3 to 0.9 mM, glutamine with a molar concentration of 1 to 3 mM, histidine with a molar concentration of 0.1 to 0.8 mM, isoleucine with a molar concentration of 0.2 to 1.0 mM, leucine with a molar concentration of 0.2 to 1.0 mM, lysine with a molar concentration of 0.2 to 1.0 mM, methionine with a molar concentration of 0.05 to 0.5 mM, phenylalanine with a molar concentration of 1.1 to 0.6 mM, proline with a molar concentration of 0.1 to 0.6 mM, serine with a molar concentration of 0.1 to 0.6 mM, threonine with a molar concentration of 0.2 to 1.0 mM, tryptophan with a molar concentration of 0.01 to 0.15 mM, tyrosine with a molar concentration of 0.1 to 0.6 mM, valine with a molar concentration of 0.2 to 1.0 mM, based on the whole cell patch cryopreservation solution.

12. The cell patch cryopreservation solution according to claim 10, wherein the nutritional additive further comprises at least one selected from a group consisting of fetal bovine serum, bovine serum albumin, human serum albumin and platelet lysate.

13. The cell patch cryopreservation solution according to claim 10, wherein the cryoprotectant is DMSO with a volume concentration of 5% and methyl cellulose with a mass concentration of 0.1 g/100 mL; the buffer system is a HEPES buffer system; the nutritional additive comprises human serum albumin with a mass concentration of 1 g/100 mL, glucose with a mass concentration of 1000 μg/mL, transferrin with a mass concentration of 1 g/mL, glycine with a molar concentration of 0.7 mM, alanine with a molar concentration of 0.3 mM, arginine with a molar concentration of 0.6 mM, aspartate with a molar concentration of 0.3 mM, aspartic acid with a molar concentration of 0.2 mM, cysteine with a molar concentration of 0.6 mM, cystine with a molar concentration of 0.1 mM, glutamic acid with a molar concentration of 0.5 mM, glutamine with a molar concentration of 2 mM, histidine with a molar concentration of 0.2 mM, isoleucine with a molar concentration of 0.4 mM, leucine with a molar concentration of 0.4 mM, lysine with a molar concentration of 0.4 mM, methionine with a molar concentration of 0.1 mM, phenylalanine with a molar concentration of 0.2 mM, proline with a molar concentration of 0.3 mM, serine with a molar concentration of 0.2 mM, threonine with a molar concentration of 0.4 mM, tryptophan with a molar concentration of 0.05 mM, tyrosine with a molar concentration of 0.2 mM, and valine with a molar concentration of 0.4 mM; the reducing agent is VC with a mass concentration of 10 μg/mL; the calcium channel inhibitor is verapamil with a molar concentration of 30 μM; the apoptosis inhibitor is the Caspase inhibitor Q-VD-OPh with a molar concentration of 10 μM, based on the whole cell patch cryopreservation solution.

14. The cell patch cryopreservation solution according to claim 10, wherein the cryoprotectant is DMSO with a volume concentration of 10%; the buffer system is a phosphate buffer system; the nutritional additive comprises bovine serum albumin with a mass concentration of 1 g/100 mL, glucose with a mass concentration of 1000 μg/mL, transferrin with a mass concentration of 1 μg/mL, glycine with a molar concentration of 0.7 mM, alanine with a molar concentration of 0.3 mM, arginine with a molar concentration of 0.6 mM, aspartic acid with a molar concentration of 0.3 mM, aspartic acid with a molar concentration of 0.2 mM, cysteine with a molar concentration of 0.6 mM, cystine with a molar concentration of 0.1 mM, glutamic acid with a molar concentration of 0.5 mM, glutamine with a molar concentration of 2 mM, histidine with a molar concentration of 0.2 mM, isoleucine with a molar concentration of 0.4 mM, leucine with a molar concentration of 0.4 mM, lysine with a molar concentration of 0.4 mM, methionine with a molar concentration of 0.1 mM, phenylalanine with a molar concentration of 0.2 mM, proline with a molar concentration of 0.3 mM, serine with a molar concentration of 0.2 mM, threonine with a molar concentration of 0.4 mM, tryptophan with a molar concentration of 0.05 mM, tyrosine with a molar concentration of 0.2 mM, and valine with a molar concentration of 0.4 mM; the reducing agent is VC with a mass concentration of 5 μg/mL and reduced glutathione with a molar concentration of 3 mM; the calcium channel inhibitor is verapamil with a molar concentration of 20 M; the apoptosis inhibitor is the Caspase inhibitor Q-VD-OPh with a molar concentration of 10 M, based on the whole cell patch cryopreservation solution.

15. The cell patch cryopreservation solution according to claim 10, wherein the cryoprotectant is DMSO with a volume concentration of 10%; the buffer system is a MOPS buffer system; the nutritional additive comprises platelet lysate with a volume concentration of 1%, glucose with a mass concentration of 1200 μg/mL, transferrin with a mass concentration of 5 μg/mL, glycine with a molar concentration of 0.7 mM, alanine with a molar concentration of 0.3 mM, arginine with a molar concentration of 0.6 mM, aspartic acid with a molar concentration of 0.3 mM, aspartic acid with a molar concentration of 0.2 mM, cysteine with a molar concentration of 0.6 mM, cystine with a molar concentration of 0.1 mM, glutamic acid with a molar concentration of 0.5 mM, glutamine with a molar concentration of 2 mM, histidine with a molar concentration of 0.2 mM, isoleucine with a molar concentration of 0.4 mM, leucine with a molar concentration of 0.4 mM, lysine with a molar concentration of 0.4 mM, methionine with a molar concentration of 0.1 mM, phenylalanine with a molar concentration of 0.2 mM, proline with a molar concentration of 0.3 mM, serine with a molar concentration of 0.2 mM, threonine with a molar concentration of 0.4 mM, tryptophan with a molar concentration of 0.05 mM, tyrosine with a molar concentration of 0.2 mM, and valine with a molar concentration of 0.4 mM; the reducing agent comprises VC with a mass concentration of 5 μg/mL, reduced glutathione with a molar concentration of 3 mM, and allopurinol with a molar concentration of 1 mM; the calcium channel inhibitor is verapamil with a molar concentration of 25 M; the apoptosis inhibitor is the P53 inhibitor Pifithrin-α with a molar concentration of 10 μM, based on the whole cell patch cryopreservation solution.

16. A method of cryopreserving a cell patch, comprising:
contacting a cell patch with the cell patch cryopreservation solution according to claim 1; and
cryopreserving the cell patch with the cell patch cryopreservation solution.

17. The method of cryopreserving the cell patch according to claim 16, wherein the cell patch cryopreservation solution is pre-cooled at 2 to 8° C. before contacting with the cell patch.

18. A composition, comprising the cell patch cryopreservation solution according to claim 1 and a cell patch.
